(19) United States
(12) Patent Application Publication
(10) Pub. No.: US 2005/0084411 A1
(43) Pub. Date: Apr. 21, 2005

(54) Method and system for producing a disinfecting solution

(76) Inventors: Harold E. Childers II, Houston, TX (US); Rudolf Matousek, Richmond, TX (US); Brent Simmons, Palo Alto, CA (US)

Correspondence Address: Jo Katherine D'Ambrosio;D'Ambrosio & Associates, Suite 465, 10260 Westheimer Road, Houston, TX, 77042, US

(21) Appl. No.: 10/690,375

(22) Filed: Oct. 21, 2003

(51) Int. Cl. A61L 2/18

(52) U.S. Cl. 422/3; 422/28; 422/37; 422/261

(57) ABSTRACT

A method and system of producing a disinfecting solution comprising directing feed water at a predetermined flow rate into a dosing storage tank; circulating the feed water from the dosing storage tank through one or more disinfectant containers and back into the dosing storage tank. Preferably, a disinfectant within the one or more disinfectant containers comprise dissolve characteristics to form a disinfecting solution when dissolved in the feed water. The flow rate of feed water directed into the dosing storage tank and circulated through out the disinfecting is predetermined and controlled to match the dissolve characteristics of the disinfectant within one or more disinfectant containers. Controlling the amount of feed water maintains a substantially consistent concentration of disinfecting solution throughout the dosing storage tank.

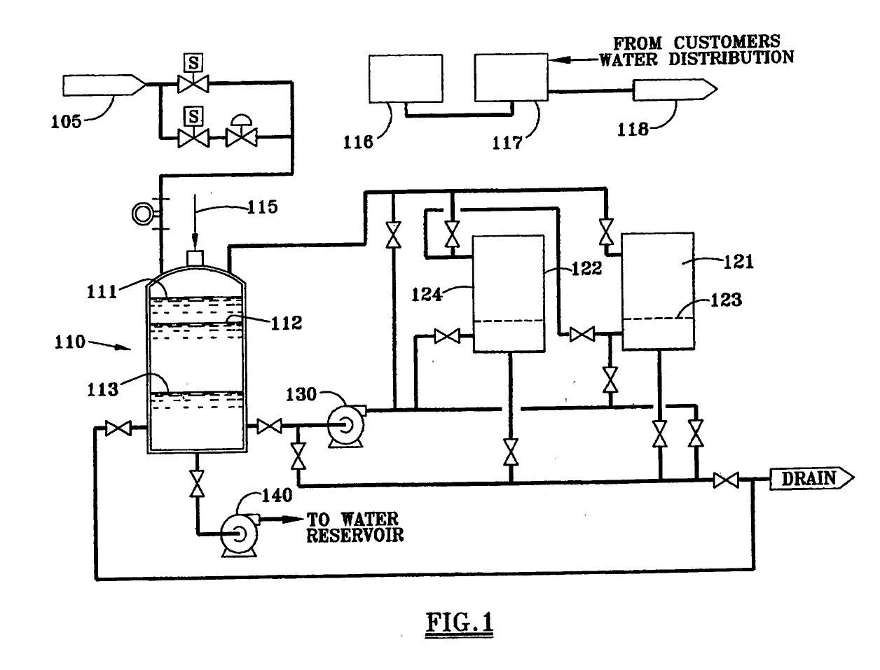

### BACKGROUND OF THE INVENTION

1. Field of the Invention

The invention relates to a method for producing a disinfecting solution. More particularly, the invention relates to a method and system for producing a disinfecting solution by controlling the flow of feed water through the system.

2. Description of the Related Art

Disinfecting agents, such as hypochlorite, are necessary to kill microorganisms present in water and waste waters to make the waters potable or at all useful for commercial and residential use. Allowed to survive, these microorganisms can be dangerous to humans who contact the contaminated water.

Hypochlorite may be added to a water stream in either a liquid or solid form. Liquid concentrated hypochlorite solution may be mixed directly with the untreated water stream to reach the desired hypochlorite concentration. Alternatively, solid hypochlorite tablets may be dissolved in the water to reach the desired Hypochlorite concentration.

It can be important to control the concentration of hypochlorite to ensure effective disinfection of the water supply and prevent the use of excess hypochlorite. Enough hypochlorite must be used to kill the dangerous microorganisms, but using excess hypochlorite wastes the expensive disinfectant.

For a liquid hypochlorite system the amount of chlorine in the treated water stream may be measured and the amount of hypochlorite solution added adjusted in response this measurement. Such adjustments may be performed manually or by an automated control system. Problems exist with liquid hypochlorite because of the frequent breakdown of hypochlorite and subsequent loss of disinfecting properties of the hypochlorite as well the difficulty of transporting and storing of the liquids. Solid hypochlorite tablet systems may control hypochlorite concentration by either manual or automatic measurement of the hypochlorite concentration of the treated stream and adjusting the volume of the untreated water into the system to achieve the desired concentration measurements. Such systems can be effective, however, they do respond to the already treated stream concentration.

Hanford, U.S. Pat. No. 3,883,429, describes an apparatus for treating contaminated potable water. The apparatus comprises a water source, a pre-filter means for removing particulate matter, two purification tanks, and an outlet. Each of the purification tanks has a recycle stream and a pressure relief valve. The second tank also includes a chemical addition means for chemically treating water in the second tank.

Calvin et al., U.S. Pat. No. 5,468,377, describes a water treatment apparatus for use with a cooling water system comprising a tank for containing a solid treatment product for treating water, a flow path means for conveying water to the tank, an outlet conduit to return water to the cooling system, and an eductor connected to the inlet conduit where water can be drained form the tank to the inlet conduit and back to the outlet conduit, and is then conveyed back to the cooling system. The solid treatment product can include a halogen.

Rossi et al., U.S. Pat. No. 6,418,958, describes a solid chemical feed system comprised of two feed bowls containing a quantity of solid liquid soluble chemicals, a valve system coupled to said feed bowls, a valve system controlling the flow of liquids into the feed bowls, a sump for receiving and storing the liquid solution, and a sensor for detecting a predetermined property of the solution and sending a signal to a control unit. The control unit responds to the signal by controlling the valve system to alternatively switch the introduction of liquid from one feed bowl to another.

Ellard et al., U.S. Pat. No. 6,129,104, describe a method for automatic dose control of a liquid treatment chemical during a treatment process, within a treatment system. In this method, chemical dosage added into the system is calculated by combining the signals from a liquid flow meter, an influent chemical concentration analyzer and/or an effluent chemical concentration analyzer. The signals are directed to a computerized dose controller that analyzes the data contained in the signals, calculates a proper dosage, and generates an output signal that controls the chemical feeder controller.

Billings, U.S. Pat. No. 5, 637,230, discusses an apparatus for chlorination of water. The apparatus includes a first chamber for receiving and holding solid calcium Hypochlorite, a second chamber connected to connected to the first, and water inlet to the first chamber to produce a chlorine solution in the second chamber. An eductor is used having a main flow passage through which the potable water flows. An eduction passage of the eductor is coupled to the second chamber of the chlorinator to educt the chlorine solution form the second chamber into the potable water. A chlorine sensor is used to sense residual chlorine levels in the potable water from the eductor. A controller responsive to the chlorine sensor controls a valve that controls the flow of potable water from the eductor. The controller attempts to maintain the residual chlorine level with a predetermined range.

Ferguson et al., U.S. Pat. No. 5,960,808, describe a method of controlling the amount of electrical conductivity increasing substances in an aqueous medium. The method involves measuring the conductivity of the aqueous medium, adding a conductivity increasing substance, and then measuring the conductivity of the mixed stream. The two conductivities are then compared and the amount of conductivity increasing substance added is adjusted in response to the level of conductivity in the mixed stream. In one preferred embodiment of the invention, the conductivity increasing substance is calcium hypochlorite.

The problem with existing tablet systems is that the concentration is not consistent as the flow of water through the tablet system varies, especially during high usage times such as storm surges.

### SUMMARY OF THE INVENTION

This invention relates to a system and method for producing a disinfecting solution for treating water, such as waste water and potable water. Advantageously, the method and system of the present invention produces a disinfecting solution using a control method that consistently produces the desired concentration of disinfectant in the solution during operation of the system instead of relying on continuous measurements of low levels of concentration in water system that has previously been treated. This method produces a more stable disinfectant concentration in the treated water stream and prevents the waste of disinfectant.

The system and method of this invention enables production of the disinfectant at a rate higher than the use rate. Because of this system and method, the response time is more rapid during peak demand periods for disinfected water, storm surges or the Super Bowl, for example.

One preferred method of producing a disinfecting solution comprises directing feed water into a dosing storage tank; circulating tank water from the dosing storage tank through one or more disinfectant containers and back into the dosing storage tank. Preferably, the one or more disinfectant containers include a disinfectant having a predetermined rate of dissolution to form a disinfecting solution when dissolved in the feed water. The amount of feed water directed into the dosing storage tank is controlled to ensure that a circulation rate of tank water does not exceed the rate of dissolution of the disinfectant. The feed water is controlled to match the known dissolve characteristics of the one or more disinfectants. Controlling the amount of feed water maintains a substantially consistent concentration of disinfectant within the disinfecting solution throughout the dosing storage tank.

In one aspect, the total volume of feed water directed to the dosing storage tank is measured. The one or more disinfectant containers comprise at least one disinfecting vessel containing dissolvable disinfectant. The addition of feed water directed to the dosing storage tank is controlled by pre-calculating the flow rate of feed water to match a predetermined dissolve rate for the dissolvable disinfectants. Matching a predetermined flow rate of feed water to a predetermined dissolve rate for the dissolvable disinfectants ensures consistency in the concentration of disinfectant within the disinfecting solution. Preferably, a specific flow rate of feed water circulated throughout the one or more disinfectant containers is maintained so as to match a predetermined dissolve rate for the dissolvable disinfectants.

In another aspect, controlling the predetermined flow rate of the feed water directed to the dosing storage tank in conjunction with controlling the specific flow rate of the flow re-circulated from the dosing storage tank through the one or more disinfectant containers and back to the dosing storage tank helps to maintain a predetermined concentration of dissolvable disinfectant material within the water in the dosing storage tank.

The dissolvable disinfectant or tablets can be comprised of a group of hypochlorites, the group selected from calcium hypochlorite, sodium hypochlorite and combinations thereof. Alternatively, the dissolvable disinfectant comprises bromine based chemicals, where necessary under extreme conditions as known in the art.

A further aspect of the method of this invention comprises controlling the flow rate of additional feed water directed to the dosing storage tank in conjunction with controlling the specific flow rate of the flow circulated from the dosing storage tank throughout the one or more disinfectant containers to maintain a predetermined concentration of dissolvable disinfectant material within the water in the dosing storage tank.

The liquid level within the dosing storage tank is monitored to determine a make-up set point within the dosing storage tank and make-up water is added to the dosing storage tank when the liquid level falls below the make-up set point.

Preferably, the method of this invention comprises determining a high level set point. When the liquid level within the dosing storage tank exceeds the high level set point, water flow and circulation of solution into the dosing storage tank are ceased as a steady state of hypochlorite solution is achieved. The method further comprises determining a low level set point, water addition and circulation within the tank begins when the fluid level within the dosing storage tank falls below the low level set point.

In still a further aspect of producing a disinfecting solution, the fluid within the dosing storage tank is re-circulated thereby preventing settling out of particulate materials within the fluid.

In one preferred method, the controlling step comprises monitoring the amount and flow rate of feed water added to the dosing storage tank, comparing the amount of feed water added to the dosing storage tank with the amount of fluid required to match the disinfecting capabilities of the disinfectants within one or more disinfectant containers, ceasing the addition of feed water to the dosing storage tank when the amount of feed water added to the dosing storage tank matches the disinfecting capabilities of the one or more disinfectant containers.

Preferably, the feed water collected within the dosing storage tank is softened water to reduce the amount of particulate material within the disinfecting solution by the system. When the disinfecting solution is produced, it is flowed or piped from the dosing storage tank to at least one user of disinfecting solution.

One preferred system for producing a disinfecting solution comprises a dosing storage tank that is fillable with a feed water, preferably, a softened water. The system further comprises one or more disinfectant containers in fluid communication with the dosing storage tank. The disinfectant container contains a dissolvable disinfectant. One or more pumps, capable of providing a re-circulating flow between the dosing storage tank and the one or more disinfectant containers, are in fluid communication with the dosing storage tank and the one or more disinfectant containers. One or more controller systems, capable of controlling the rate of feed water flow directed into the dosing storage tank to match the dissolve rate of the disinfectant are placed within the system of this invention. One preferred system further comprises a level controller capable of monitoring the liquid level within the dosing storage tank. The level controller is in operative communication with the controller system and the system further comprises a high level set point and a low level set point.

The foregoing has outlined rather broadly several aspects of the present invention in order that the detailed description of the invention that follows may be better understood and thus is not intended to narrow or limit in any manner the appended claims which define the invention. Additional features and advantages of the invention will be described hereinafter, which form the subject of the claims of the invention. It should be appreciated by those skilled in the art that the conception and the specific embodiment disclosed might be readily utilized as a basis for modifying the composition or method of manufacture for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

The FIGURE illustrates a schematic of the method and system of the invention.

### DESCRIPTION OF THE PREFERRED EMBODIMENTS

The method and system of this invention produces a disinfecting solution that is used to treat water by killing off microorganisms that are present so that the water is potable or, at least sufficiently low in microorganism count to be useful for either commercial or residential use. Advantageously, the system and method of this invention enables production of the disinfectant at a rate higher than the use rate so that the response time is more rapid during peak demand periods

Applicants' method of producing a disinfecting solution directs the contaminated or feed water into a dosing storage tank for circulation through one or more disinfectant containers. The dosing feed tank can vary in size depending on the desired amount and concentration of disinfecting solution to be produced. Storage tanks for this purpose are available in various sizes including 750 gallon capacities and 1500 gallon capacities. Smaller or larger tanks are possible under the method of this invention. The 750 gallon and 1500 gallon capacity tanks will be used for purposes of this description.

Feed water from the dosing storage tank is circulated through one or more disinfectant containers and back into the dosing storage tank. Preferably, disinfectants within the one or more the disinfectant containers comprise dissolve characteristics to form a disinfecting solution when dissolved in the feed water. The one or more disinfectant containers comprise at least one disinfecting vessel containing dissolvable disinfectant, preferably dissolvable tablets. The tablets can be stacked in one or more tablet feeder (dissolve) tubes and tablet loads can vary depending on the disinfecting requirements of the end user. Loading of the dissolve tube can be done in batches that match industry standard tablets packaged in weights of 50 lb and 100 lb containers. In one preferred method, the maximum tablet load is about 287 tablets for use in a 750 gallon tank; this load produces from about 25 PPD (pounds per day) of chlorine equivalent to about 50 PPD of chlorine equivalent. A larger dosing storage tank, a 1500 gallon tank for example, can accommodate about 574 tablets so that the output increases from 50 PPD of chlorine equivalent to about 100 PPD of chlorine equivalent. The dissolvable disinfectant is preferably comprised of a group of hypochlorites, the group selected from calcium hypochlorite, sodium hypochlorite and combinations thereof. Alternatively, the dissolvable disinfectant comprises bromine based chemicals depending on the needs of the user.

Initially, a pressurized water source, such as a feed water pump or a water tower, directs the feed water to the dosage storage tank. When the tank is filled to a minimum or low level to ensure that a water circulation pump is flooded, the circulating pump is activated to circulate the tank water through the one or more disinfectant containers. The liquid level in the storage tank is monitored by an ultrasonic level sensor/transmitter. The flow rate of feed water directed into the dosing storage tank is controlled to match the dissolve characteristics of the disinfectants within the one or more disinfectant containers and ensure that a circulation rate of tank water does not exceed the rate of dissolution of the disinfectant. The dissolve rate of the tablets is predetermined and based on empirical data and predetermined based on the physical and chemical characteristics of the tablets being used, such as size, density, age and design of tablets. The desired flow rate is then calculated to produce a specific concentration of disinfectant within solution. Controlling the flow rate of feed water maintains a substantially consistent concentration of disinfecting solution throughout the dosing storage tank.

The predetermined flow rate of the feed water directed to the dosing storage tank is controlled in conjunction with controlling the specific flow rate of the flow circulated from the dosing storage tank through the one or more disinfectant containers and back to the dosing storage tank. Preferably, this specific flow rate of circulated water is predetermined and maintained throughout the operation of the disinfecting system to match the predetermined dissolve rate of the dissolvable disinfectants thereby ensuring that the circulation rate of tank water does not exceed the rate of dissolution of the disinfectant. Matching the specific flow rate to the dissolve rate of the disinfectant allows a predetermined concentration of dissolvable disinfectant to be maintained throughout the system.

In another aspect, the total volume of feed water directed to the dosing storage tank is measured and, in one preferred embodiment, calculated to provide enough disinfecting solution for a 24 period. The addition of feed water directed to the dosing storage tank is controlled by matching a predetermined flow rate of feed water to the predetermined dissolve rate for the dissolvable disinfectants. Preferably, the feed water collected within the dosing storage tank is softened water to reduce the amount of particulate material produced disinfecting solution produced by the system.

As the disinfecting solution circulates through the dissolve system, the tablets are dissolved. Additional feed water, at a predetermined flow rate, is directed to the dosing storage tank to maintain the desired concentration of disinfectant material; this process is controlled by matching the predetermined flow rate of feed water to a predetermined dissolve rate for the dissolvable disinfectants. The dissolvable disinfectant or tablets can be comprised of a group of hypochlorites, the group selected from calcium hypochlorite, sodium hypochlorite and combinations thereof. Alternatively, the dissolvable disinfectant comprises bromine based chemicals, where necessary under extreme conditions as known in the art.

In one embodiment of this invention, the concentration of the hypochlorite is maintained within a range from about 0.2% by weight hypochlorite to about 10.0% by weight of hypochlorite in the disinfected solution available to the user (except during the initial start up of the system. Alternatively, the concentration of the hypochlorite is maintained at about 0.9% by weight of hypochlorite solution in the disinfected solution available to the user. When the disinfecting solution is produced, it flows or is piped from the dosing storage tank to at least one user of disinfecting solution.

A further aspect of the method of this invention comprises using additional or make up water to replace the disinfecting solution depleted from the system by the user. A further innovated aspect of this invention is controlling the flow rate of make up feed water directed to the dosing storage tank during the operation of the disinfecting system in conjunction with controlling the specific flow rate of the tank water circulated from the dosing storage tank throughout the one or more disinfectant containers. Controlling the flow rate of the initial feed water, the water addition or make up water flow rate, and the flow rate of the circulated disinfectant solution through the dissolve system, to match a predetermined concentration of dissolvable disinfectant material within the water, maintains a consistency of concentration of the disinfecting solution throughout the tank and directed to the user.

The operation of the system of this invention typically starts with a full stack of disinfecting tablets within the dissolve tube. A water solenoid valve fills the storage tank to a minimum or low level to ensure that the water circulation pump suction is flooded. When the level is satisfied, the solenoid valve will remain open and continue adding water to the storage tank at a fixed flow rate, pre-determined to match the dissolve rate of the disinfecting tablets. In one preferred method, the fixed flow rate to the dosing storage tank is about 1 gallon per minute. The circulation pump circulates water through the dissolve system at a faster flow rate, also predetermined to maintain a specific concentration of disinfectant within the solution. One preferred water flow rate for circulation of solution through the dissolve system is maintained at about 20 gallons per minute. The flow rate of the make-up feed water directed to the dosing storage tank is predetermined to match the dissolve rate of the disinfectant, preferably at about 1 gallon per minute and the flow rate of the water circulated through the one or more disinfectant containers is a different, predetermined flow rate also calculated based on the dissolve rate of the disinfectant.

As disinfecting solution is depleted upon demand by a user's dosing system, the liquid level within the dosing storage tank is monitored to determine a make-up set point within the dosing storage tank. Make-up feed water is added to the dosing storage tank when the liquid level falls below the make-up set point, preferably about ⅔ of a high level set point. The high level set point is determined and the feed water flow into the dosing storage tank is stopped when the liquid level within the dosing storage tank exceeds the high level set point. A low level set point is also determined and circulation from the dosing storage tank to the dissolve system ceases when the fluid level within the dosing storage tank falls below the low level set point and make-up feed water is added to the storage tank.

During the system's cycle of operation various sensors, flow meters and controllers measure the flow rates throughout the system and control the flow rates to match predetermined amounts. A Programable Logic Controller (PLC) receives input from one or more ultrasonic level sensors and transmitters and flow meters to monitor the level of solution in the dosing tank, control the circulation pump and activate the flow valves such as a make-up water addition solenoid valve. Once activated, the circulation pump will circulate the disinfecting solution through the dissolve system at a specific flow rate to maintain the concentration of the disinfectant, hypochlorite for example, within the solution.

In another aspect of this invention, the fluid within the dosing storage tank is re-circulated within the dosing tank thereby preventing settling out of particulate materials within the fluid.

In another embodiment of the method of producing a disinfecting solution feed water is directed to a dosing storage tank. The water within the dosing storage tank is circulated from the dosing storage tank through one or more disinfectant containers and back into the dosing storage tank, wherein the one or more disinfectant containers includes a dissolvable disinfectant. The amount of feed water added to the dosing storage tank is monitored. The amount of feed water added to the dosing storage tank is then compared with the amount of liquid required to match the disinfecting capabilities of the disinfectant within the one or more disinfectant containers. The addition of feed water to the dosing storage tank is ceased when the amount of feed water added to the dosing storage tank matches the disinfecting capabilities of the disinfectant within the one or more disinfectant containers.

The flow rate of the feed water directed to the dosing storage tank is controlled in conjunction with controlling the flow rate of the flow circulated from the dosing storage tank through the one or more disinfectant containers so as to match a predetermined dissolve rate of dissolvable disinfectant material within the solution in the dosing storage tank.

In another embodiment of this invention, a system for producing a disinfecting solution 100 comprises a dosing storage tank 110 that is fillable with a feed water from a pressurized water supply 105, preferably, a softened water. The system 100 further comprises one or more disinfectant containers 120, 121 in fluid communication with the dosing storage tank 110. The one or more disinfectant containers 120, 121 contains a dissolvable disinfectant, preferably dissolvable tablets 123 contained within a dissolve tube 124. One or more pumps 130, capable of providing a re-circulating flow between the dosing storage tank 110 and the one or more disinfectant containers 120, 121, are in fluid communication with the dosing storage tank 110 and the one or more disinfectant containers 120, 121. One or more controller systems 116, capable of controlling the rate of feed water flow directed into the dosing storage tank 110 to match the dissolve rate of the disinfectant 123 are placed within the system 100 of this invention. One preferred system further comprises a level controller 115 capable of monitoring the liquid level within the dosing storage tank 110. The level controller 115 is in operative communication with the controller system 116 and the system 100 further comprises a high level set point 111, a low level set point 113, and a make up water set point 112. The make up water set point is preferably at a point which is about two thirds capacity of the storage tank 110.

The dissolvable disinfectant is selected from a group of disinfectants comprising calcium hypochlorite, sodium hypochlorite, and combinations thereof. An effluent stream is directed to a user by means of a dosing pump. Depending on the requirements of a user, the effluent stream comprises from about 0.2% by weight of hypochlorite to about 10.0% by weight of hypochlorite.

Test Data

Following are dissolve curves for three testing experiments.

In test number one, see graft dated Jul. 16, 2003, attached, a 50 lb batch of dissolve tablets had a dissolve rate of 1.9# (tablets) per hour resulting in a one weight percent solution in the dosing tank. The volume of this tank was 350 gallons. The concentration of disinfectant yielded was 10.3 gpl chlorine achieved in 12.8 hours.

Test number two, see graft dated Jul. 17, 2002, attached, again using a 50 lb batch of dissolve tablets having a dissolve rate of 1.6# (tablets) per hour resulted in a 4 weight percent solution. The volume of this tank was 70 gallons. The concentration of disinfectant yielded was 51.4 gpl chlorine achieved in 15.2 hours.

The third test, see graph dated Jul. 21, 2002, attached, used 100 lb batch of dissolve tablets having a dissolve rate of 3.8# (tablets) per hour resulted in a one weight percent solution in the dosing tank. The volume of this tank was 800 gallons. The concentration of disinfectant yielded was 9 gpl chlorine achieved in 11.5 hours or 9.75 gpl chlorine achieved in 12.8 hours .

[image]

[image]

It appears that doubling the number of tablets at the same concentration doubles the dissolve rate, therefore the water addition or make-up flow rate must be doubled to maintain a constant concentration in the tank.

The flow rates in each of the tests were consistent through out the time period of testing.

Although the present invention and its advantages have been described in detail, it should be understood that various changes, substitutions and alterations can be made herein without departing from the spirit and scope of the invention as defined by the appended claims.

1. A method of producing a disinfecting solution comprising:
(a) directing feed water into a dosing storage tank;
(b) circulating the tank water from the dosing storage tank through one or more disinfectant containers and back into the dosing storage tank, the one or more disinfectant containers including a disinfectant with a predetermined rate of dissolution;
(c) controlling the amount of feed water directed into the dosing storage tank to ensure that a circulation rate of the tank water does not exceed the rate of dissolution of the disinfectant; and
(d) maintaining a substantially consistent concentration of disinfectant throughout the dosing storage tank.

2. The method of claim 1, wherein step (c) further comprises measuring the total volume of feed water directed to the dosing storage tank.

3. The method of claim 1 wherein the one or more disinfectant containers comprises at least one disinfecting vessel containing dissolvable disinfectant.

4. The method of claim 1 further comprising the step of controlling the addition of feed water directed to the dosing storage tank by a pre-calculating the flow rate of feed water to match a predetermined dissolve rate for the dissolvable disinfectants.

5. The method of claim 4, wherein step (d) further comprises maintaining a specific flow rate of feed water circulated throughout the one or more disinfectant containers to match a predetermined dissolve rate for the dissolvable disinfectants.

6. The method of claim 5 further comprising controlling the predetermined flow rate of the feed water directed to the dosing storage tank in conjunction with controlling the specific flow rate of the flow circulated from the dosing storage tank through the one or more disinfectant containers and back to the dosing storage tank to maintain a predetermined concentration of dissolvable disinfectant material within the water in the dosing storage tank.

7. The method of claim 4, wherein the dissolvable disinfectant comprises dissolvable tablets.

8. The method of claim 4, wherein the dissolvable disinfectant is comprised of a group of hypochlorites, the group selected from calcium hypochlorite, sodium hypochlorite and combinations thereof.

9. The method of claim 4, wherein the dissolvable disinfectant comprises bromine based chemicals, where necessary.

10. The method of claim 4 further comprising controlling the flow rate of additional feed water directed to the dosing storage tank in conjunction with controlling the specific flow rate of the flow circulated from the dosing storage tank throughout the one or more disinfectant containers to maintain a predetermined concentration of dissolvable disinfectant material within the water in the dosing storage tank.

11. The method of claim 1 further comprising monitoring the liquid level within the dosing storage tank.

12. The method of claim 11 further comprising determining a make-up set point within the dosing storage tank and adding make-up water to the dosing storage tank when the liquid level within the dosing storage tank falls below the make-up set point.

13. The method of claim 10 further comprising determining a high level set point and ceasing water flow into the dosing storage tank when the liquid level within the dosing storage tank exceeds the high level set point.

14. The method of claim 10 further comprising determining a low level set point and ceasing circulation from the dosing storage tank when the fluid level within the dosing storage tank falls below the low level set point.

15. The method of claim 1 re-circulating the fluid within the dosing storage tank thereby preventing settling out of particulate materials within the fluid.

16. The method of claim 1, wherein the controlling step comprises monitoring the amount and flow rate of feed water added to the dosing storage tank, comparing the amount of feed water added to the dosing storage tank with the amount of fluid required to match the disinfecting capabilities of the disinfectant within the one or more disinfectant containers, ceasing the addition of feed water to the dosing storage tank when the amount of feed water added to the dosing storage tank matches the disinfecting capabilities of the disinfectant within the one or more disinfectant containers.

17. The method of claim 16 further comprising recharging the disinfecting capabilities of the disinfectant within the one or more disinfectant containers.

18. The method of claim 1 wherein the feed water collected within the dosing storage tank is softened water.

19. The method of claim 1 further comprising maintaining from about 0.2% by weight hypochlorite to about 10.0% by weight of hypochlorite in the disinfected solution within the dosing storage tank.

20. The method of claim 1 further comprising maintaining about 0.9% by weight of hypochlorite solution in the disinfected solution within the dosing storage tank.

21. The method of claim 1 further comprising flowing a disinfecting solution from the dosing storage tank to at least one user of disinfecting solution.

22. A method of producing a disinfecting solution comprising:
(a) directing feed water into a dosing storage tank;
(b) circulating the tank water from the dosing storage tank through one or more disinfectant containers and back to the dosing storage tank, wherein the one or more disinfectant containers contains a dissolvable disinfectant;
(c) controlling the flow rate of the feed water directed to the dosing storage tank and the flow rate of the tank water circulating from the dosing storage tank through one or more disinfectant containers to match a predetermined dissolve rate of the disinfectant.

23. The method of claim 22 further comprising flowing a disinfecting solution from the dosing storage tank to at least one user of a disinfecting solution.

24. The method of claim 22 further comprising monitoring the liquid level within the dosing storage tank.

25. The method of claim 24 further comprising determining a make-up set point within the dosing storage tank and adding make-up water to the dosing storage tank when the liquid level within the dosing storage tank falls below the make-up set point.

26. The method of claim 24 further comprising determining a high level set point and ceasing water flow into the dosing storage tank when the liquid level within the dosing storage tank exceeds the high level set point.

27. The method of claim 22 further comprising determining a low level set point and ceasing circulation from the dosing storage tank when the fluid level within the dosing storage tank falls below the low level set point.

28. The method of claim 22 wherein the flow rate of the additional feed water directed to the dosing storage tank is predetermined to match the dissolve rate of the disinfectant and the flow rate of the water circulated through the one or more disinfectant containers is a different predetermined flow rate.

29. The method of claim 22 further comprising circulating the fluid within the dosing storage tank thereby preventing settling out of particulate materials within the fluid.

30. The method of claim 22, further comprising controlling the flow rate of the feed water directed to the dosing storage tank in conjunction with controlling the flow rate of the flow circulated from the dosing storage tank through the one or more disinfectant containers to maintain a predetermined concentration of dissolvable disinfectant material within the water in the dosing storage tank.

31. The method of claim 22, further comprising the step of directing an effluent stream to a user, the effluent stream comprising about 0.2 weight percent of a hypochlorite solution to about 10.0 weight percent of a hypochlorite solution.

32. The method of claim 22, wherein the dissolvable material is selected from a group comprising calcium hypochlorite, sodium hypochlorite, and a combination thereof.

33. The method of claim 22 wherein the feed water directed to the dosing storage tank comprises softened water.

34. A method of producing a disinfecting solution comprising:
(a) directing feed water to a dosing storage tank;
(b) circulating the water within the dosing storage tank from the dosing storage tank through one or more disinfectant containers and back into the dosing storage tank, wherein the one or more disinfectant containers includes a dissolvable disinfectant;
(c) monitoring the amount of feed water added to the dosing storage tank;
(d) comparing the amount of feed water added to the dosing storage tank with the amount of fluid required to match the disinfecting capabilities of the disinfectant within the one or more disinfectant containers;
(e) ceasing the addition of feed water to the dosing storage tank when the amount of feed water added to the dosing storage tank matches the disinfecting capabilities of the disinfectant within the one or more disinfectant containers;
(f) controlling the flow rate of the feed water directed to the dosing storage tank in conjunction with controlling the flow rate of the flow circulated from the dosing storage tank through the one or more disinfectant containers to match a predetermined dissolve rate of dissolvable disinfectant material within the solution in the dosing storage tank to ensure that the circulation rate of the tank water does not exceed the rate of dissolution of disinfectant.

35. The method of claim 34 further comprising, flowing the disinfecting solution from the dosing storage tank to at least one user of disinfected water.

36. The method of claim 34 further comprising monitoring the liquid level within the dosing storage tank.

37. The method of claim 36 further comprising determining a make-up set point within the dosing storage tank and adding make-up water to the dosing storage tank when the liquid level within the dosing storage tank falls below the make-up set point.

38. The method of claim 36 further comprising determining a high level set point and ceasing water flow into the dosing storage tank when the liquid level within the dosing storage tank exceeds the high level set point.

39. The method of claim 36 further comprising determining a low level set point and ceasing circulation from the dosing storage tank when the fluid level within the dosing storage tank falls below the low level set point.

40. The method of claim 39, wherein the disinfectant comprises dissolvable tablets.

41. The method of claim 34 further comprising circulating the fluid within the dosing storage tank thereby preventing settling out of particulate materials within the fluid.

42. The method of claim 34 wherein the feed water directed to the dosing storage tank is softened water.

43. A method of producing a disinfecting solution comprising:
(a) directing feed water into a dosing storage tank, the feed water comprising softened water;
(b) circulating tank water from the dosing storage tank through one or more disinfectant containers and back to the dosing storage tank, wherein the one or more disinfectant containers contains a dissolvable disinfectant;
(c) controlling the flow rate of the feed water directed to the dosing storage tank to match a predetermined dissolve rate of the disinfectant.
(d) re-circulating the fluid within the dosing storage tank thereby preventing settling out of particulate materials within the fluid.

44. A system for producing a disinfecting solution comprising:
a dosing storage tank fillable with a feed water;
one or more disinfectant containers comprising a dissolvable disinfectant, the one or more disinfectant containers in fluid communication with the dosing storage tank;
a pump in fluid communication with the dosing storage tank and the one or more disinfectant containers, the pump capable of providing a circulating flow between the dosing storage tank and the one or more disinfectant containers; and
a controller system capable of controlling the rate of feed water flow directed into the dosing storage tank and the rate of tank water circulated to the disinfectant container to match the dissolve rate of the disinfectant.

45. The system of claim 44 wherein the flow rate of feed water into the dosing storage tank differs from the flow rate of the tank water circulating to and from the disinfectant container.

46. The system of claim 44 wherein the feed water comprises softened water.

47. The system of claim 44 further comprising a level controller capable of monitoring the liquid level within the dosing storage tank.

48. The system of claim 47 wherein the level controller is in operative communication with the controller system and the system further comprises a high level set point and a low level set point.

49. The system of claim 44, wherein the dissolvable disinfectant is selected from a group of disinfectants comprising calcium hypochlorite, sodium hypochlorite, and combinations thereof.

50. The system of claim 44 comprising an effluent stream directed to a user, the effluent stream comprising from about 0.2% by weight of hypochlorite to about 10.0% by weight of hypochlorite.

51. The system of claim 44 comprising an effluent stream directed to a user, the effluent stream comprising about 0.9% by weight of hypochlorite.
